(19) United States
(12) Patent Application Publication
(10) Pub. No.: US 2006/0085058 A1
(43) Pub. Date: Apr. 20, 2006

(54) System and method for delivering a biologically active material to a body lumen

(76) Inventors: Arthur L. Rosenthal, Boston, MA (US); James J. Barry, Marlborough, MA (US)

Correspondence Address: JONES DAY, 222 EAST 41ST ST, NEW YORK, NY, 10017, US

(21) Appl. No.: 10/969,664

(22) Filed: Oct. 20, 2004

(51) Int. Cl. A61F 2/06

(52) U.S. Cl. 623/1.11

(57) ABSTRACT

A medical device system that is insertable in a body lumen for treating and/or preventing injury to a body lumen wall is disclosed. The system comprises a stent comprising a sidewall having a first edge and a second edge, and a balloon capable of being disposed within the sidewall. The balloon has a first end portion that is capable of contacting the first edge of the sidewall and a second end portion that is capable of contacting the second edge of the sidewall when the balloon is expanded. At least the first end portion of the balloon comprises a first biologically active material. When the balloon is expanded, the first end portion of the balloon extends axially beyond the first edge of the sidewall in order to deliver the first biologically active material to a portion of the body lumen wall that is adjacent to the first edge of the sidewall. Methods for deploying the system are also disclosed.

### FIELD OF THE INVENTION

This invention relates generally to medical device systems that are inserted into the body lumen of a patient. More particularly, the invention is directed to systems including a stent and a balloon comprising a biologically active material, and method of making such systems. The present invention is also directed to methods of delivering a biologically active material to a body lumen using such systems.

### BACKGROUND OF THE INVENTION

Balloon angioplasty has been very effective in treating stenosis, i.e., to open blocked vessels and restore normal levels of blood flow. However, although once a blocked vessel is opened, the treated vessel can restenose, i.e., reclose, shortly after the procedure. Thus, patients may have to undergo repeated angioplasty or even surgery.

Implantable stent prostheses or stents are used to reduce restenosis after balloon angioplasty or other procedures using catheters. A stent in the form of a wire mesh tube props open an artery that has recently been cleared using angioplasty. A balloon expandable stent is collapsed to a small diameter, placed over an angioplasty balloon catheter and moved into the area of the blockage. When the balloon is inflated, the stent expands, locks in place and forms a scaffold to hold the artery open. A self-expandable stent is collapsed to a small diameter by placing the stent in a sheath, and expands in the area of the blockage when the sheath surrounding the stent is removed. Usually, the stent stays in the artery permanently, holds it open, improves blood flow to the heart muscle and relieves symptoms. The stent procedure is fairly common, and various types of stents have been developed and actually used.

To reduce the possibility of restenosis and also to locally deliver a biologically active material in a patient's lumen, various types of biologically active material-coated expandable stents have been proposed for angioplasty and localized delivery of the biologically active material to a body lumen. See, e.g., U.S. Pat. No. 6,099,562 to Ding et al. The biologically active material is released from the coated stent.

However, the concentration of the biologically active material released in the body tissue surrounding the stent may not be uniform. For instance, the body tissue in proximity to an edge of the stent is exposed to a lower concentration of the biologically active material than the body tissue in proximity to the middle portion of the stent.

Also, recent data shows that restenosis occurs at the edges of the stents about five times more often than at the middle portion of stents, i.e., the “edge effect”. The “edge effect” may be caused by the lesser concentration of biological active material that is present in body tissue in proximity to the edges of the stent. Also, the pressure or stress that the stent exerts against the surrounding tissue is concentrated at the edges of the stent. Such concentrated stress may also contribute to the “edge effect”. Therefore, there is a need for a way to reduce the “edge effect.” One way to reduce the “edge effect,” is to have a medical device having a structure wherein the stress exerted against the body tissue in proximity to the edges of the stent is reduced and/or such body tissue is exposed to a greater amount of biologically active material.

Furthermore, when a balloon and a balloon expandable stent disposed on the balloon are expanded, the stent does not extend to the ends of the balloon, i.e., the ends of the stent do not cover the entire balloon's length. Thus, the balloon inflates beyond the ends of the stents, and the portions of the balloon beyond the stents' ends directly contact the patient's lumen wall. Such direct contact with the balloon may cause a tissue injury in the patient's lumen wall. This injury may ultimately lead to restenosis in the areas of the body lumen adjacent the ends of the stent. However, to reduce such injury by using a balloon having a length which is matched exactly to a stent length is impractical because: (1) it is difficult to align the stent with the balloon during crimping; (2) both stent and balloon are manufactured within a small but finite tolerance that provides a range of component sizes; and (3) stents will shortened during expansion. Therefore, there is a need for a medical device system having a structure that reduces and/or treats such injury caused by the ends of a balloon.

Therefore, there is a need for a medical device system that can deliver a therapeutic substance to the areas of a body lumen wall adjacent to the edges of a stent that is deployed within the body lumen, without causing additional trauma to the body lumen wall. There is also a need for a method of making and deploying such a device.

### SUMMARY OF THE INVENTION

These and other objectives are accomplished by the present invention. To achieve the aforementioned objectives, we have invented medical device systems for treating a body lumen wall of a body lumen and methods of making such systems. The medical device system includes a stent that is insertable into a body lumen having a body lumen wall, wherein the stent comprises a sidewall having a first edge and a second edge; and a balloon capable of being disposed within the stent sidewall. The balloon has an outer surface, a middle portion, a first end portion that is capable of contacting the first edge of the stent sidewall when the balloon is expanded, and a second end portion that is capable of contacting the second edge of the stent sidewall when the balloon is expanded. At least the first end portion of the balloon comprises a first biologically active material. Also, the balloon is capable of being expanded in the body lumen so that the first end portion of the balloon extends axially beyond the first edge of the stent sidewall in order to deliver the first biologically active material to a portion of the body lumen wall that is adjacent to the first edge of the stent sidewall.

In certain embodiments, the first end portion of the balloon comprises pores through which the biologically active material can be delivered to the body lumen wall.

In other embodiments, the balloon comprises a balloon coating disposed on the outer surface of at least the first end portion of the balloon, and the balloon coating comprises the first biologically active material such as, for example, paclitaxel, a paclitaxel analogue, a paclitaxel derivative, or a combination thereof. The balloon coating may further comprise a polymer such as a biostable polymer capable of releasing the biologically active material including, but not limited to, a hydrogel or phosphoryl choline.

In other embodiments, the second end portion of the balloon also comprises the first biologically active material, and the balloon is capable of being expanded so that the second end portion of the balloon extends axially beyond the second edge of the stent sidewall in order to deliver the first biologically active material to a portion of the body lumen wall that is adjacent to the second edge of the stent sidewall.

In certain other embodiments, the stent sidewall further comprises a surface and a stent coating disposed on at least a portion of the surface of the stent sidewall. The stent coating may comprise a second biologically active material such as an antiproliferative agent. The stent coating can also include a polymer.

Another embodiment of the present invention is directed to a medical device system for treating a body lumen wall of a body lumen comprising: a stent that is insertable into a body lumen having a body lumen wall, wherein the stent comprises a sidewall having a surface, a first edge and a second edge, and a stent coating disposed on at least a portion of the surface of the stent sidewall; and a balloon capable of being disposed within the stent sidewall. The balloon has an outer surface, a middle portion, a first end portion that is capable of contacting the first edge of the stent sidewall when the balloon is expanded and a second end portion that is capable of contacting the second edge of the stent sidewall when the balloon is expanded. At least the first end portion of the balloon and the second end portion of the balloon both comprise a first biologically active material, and the balloon is capable of being expanded so that the first end portion of the balloon extends axially beyond the first edge of the sidewall and the second end portion of the balloon extends axially beyond the second edge of the stent sidewall in order to deliver the first biologically active material to portions of the body lumen wall that are adjacent to the first edge and the second edge of the stent sidewall, respectively.

The first end portion of the balloon and the second end portion of the balloon may both comprise pores through which the biologically active material can be delivered to the body lumen wall. The balloon may further comprise a balloon coating disposed on the outer surface of the first end portion and the second end portion of the balloon, wherein the balloon coating comprises the first biologically active material. The first biologically active material may comprise paclitaxel, a paclitaxel analogue, a paclitaxel derivative, or a combination thereof.

The balloon coating may further comprise a polymer. For example, the polymer may comprise a biostable polymer capable of releasing the biologically active material, such as a hydrogel or phosphoryl choline.

In this embodiment, the stent coating may further comprise a second biologically active material, such as an antiproliferative agent. Also, the stent coating may further comprise a polymer.

In yet another embodiment, the medical device system of the present invention comprises: a stent that is insertable into a body lumen having a body lumen wall, wherein the stent comprises a sidewall having a first edge and a second edge; and a balloon capable of being disposed within the stent sidewall. The balloon has an outer surface, a middle portion, a first end portion that is capable of contacting the first edge of the stent sidewall when the balloon is expanded, and a second end portion that is capable of contacting the second edge of the stent sidewall when the balloon is expanded. At least the first end portion of the balloon comprises a first biologically active material, and the balloon is capable of being expanded so that the first end portion of the balloon extends axially beyond the first edge of the stent sidewall in order to deliver the first biologically active material to a portion of the body lumen wall that is adjacent to the first edge of the stent sidewall. In this embodiment, the middle portion of the balloon is substantially free of a biologically active material.

In certain embodiments, the first end portion of the balloon comprise pores through which the biologically active material can be delivered to the body lumen wall.

In other embodiments, the balloon further comprises a balloon coating disposed on the outer surface of at least the first end portion of the balloon, and the balloon coating comprises the first biologically active material. Suitable first biologically active materials include, for example, paclitaxel, a paclitaxel analogue, a paclitaxel derivative, and combinations thereof. The balloon coating may further comprise a polymer such as a biostable polymer capable of releasing the biologically active material. For example, the polymer may comprise a hydrogel or phosphoryl choline.

In another embodiment, the second end portion of the balloon comprises the first biologically active material, and the balloon is capable of being expanded so that the second end portion of the balloon extends axially beyond the second edge of the stent sidewall in order to deliver the first biologically active material to a portion of the body lumen wall that is adjacent to the second edge of the stent sidewall.

The stent sidewall may further comprise a surface, wherein a stent coating is disposed on at least a portion of the surface of the stent sidewall. The stent coating may comprise a second biologically active material such as an antiproliferative agent. The stent coating may also include a polymer.

In another embodiment, the present invention is directed to a method of making a medical device system for treating a body lumen wall of a body lumen. This method includes providing a stent that is insertable into a body lumen having a body lumen wall, wherein the stent comprises a sidewall having a first edge and a second edge; and disposing a balloon within the sidewall. The balloon has an outer surface, a middle portion, a first end portion that is capable of contacting the first edge of the stent sidewall when the balloon is expanded and a second end portion that is capable of contacting the second edge of the stent sidewall when the balloon is expanded. The first end portion of the balloon comprises a first biologically active material, and the balloon is capable of being expanded so that the first end portion of the balloon extends axially beyond the first edge of the stent sidewall in order to deliver the first biologically active material to a portion of the body lumen wall that is adjacent to the first edge of the stent sidewall.

In certain embodiments, the second end portion of the balloon comprises the first biologically active material, and the balloon is capable of being expanded so that the second end portion of the balloon extends axially beyond the second edge of the stent sidewall in order to deliver the first biologically active material to a portion of the body lumen wall that is adjacent to the second edge of the stent sidewall.

Thus, the present invention provides for a medical device system that can deliver a therapeutic substance to the areas of a body lumen wall adjacent to the ends of a stent that is deployed within the body lumen, without causing additional trauma to the body lumen wall. The present invention also provides a method for making and delivering such a medical device system.

### BRIEF DESCRIPTION OF THE DRAWINGS

FIG. 1 is a side view of a medical device system of the present invention.

FIG. 2 is a cross-sectional side view of the medical device system of FIG. 1 within a body lumen.

FIG. 3 is a cross-sectional side view of a medical device system of the present invention deployed within a body lumen in which the first end portion and second end portion of the balloon are different lengths.

FIG. 4 is a cross-sectional side view of a medical device system of the present invention deployed within a body lumen in which the first end portion of the balloon is coated with a balloon coating.

FIG. 5 is a cross-sectional side view of a medical device system of the present invention deployed within a body lumen in which the first end portion and the second end portion are coated with a balloon coating.

FIG. 6 is a cross-sectional side view of a medical device system of the present invention deployed within a body lumen in which the first end portion and the second portion of the balloon comprise pores through which a biologically active material is delivered to the body lumen wall.

FIG. 7 is a cross-sectional side view of a medical device system of the present invention deployed within a body lumen in which the first end portion and the second end portion are coated with a balloon coating and a surface of the sidewall of the stent is coated with a stent coating.

### DETAILED DESCRIPTION OF THE INVENTION

The medical device system of the present invention is insertable into a body lumen of a patient for treating a body lumen wall.

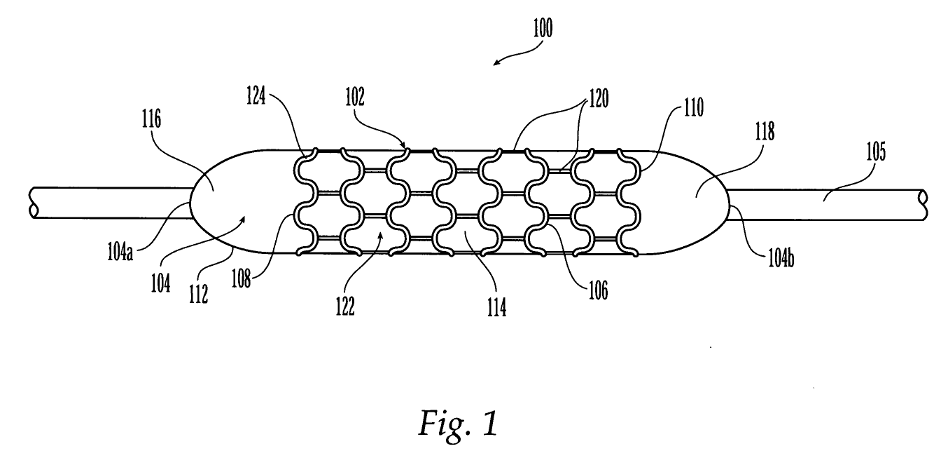

FIG. 1 is a side view of a medical device system of the present invention. The medical device system 100 generally includes a stent 102 disposed on a balloon 104 which is disposed on a catheter 105 as shown in FIG. 1. The stent 102 has a sidewall 106 having a first edge 108 and second edge 110 which correspond to the proximal and distal ends of the stent.

The stent 102 preferably has a plurality of openings 122 therein. For example, the sidewall 106 of the stent 102 may be made of a plurality of struts 120 that form a plurality of openings 122 in the sidewall of the stent 102 as shown in FIG. 1. Stents with other designs may also be used. The sidewall 106 of the stent 102 also has an outer surface 124 that is exposed to a body lumen wall when the stent 102 is inserted into the body lumen. When the stent 102 is comprised of struts 120 as shown in FIG. 1, the outer surface 124 of the stent sidewall 124 refers to the surfaces of the struts 120 that are to directly contact the body lumen or tissue.

As shown in FIG. 1, the balloon 104 of the system 100 of the present invention has an outer surface 112, a middle portion 114, a first end portion 116 which is capable of contacting the first edge 108 of the sidewall 106 when the balloon is expanded and a second end portion 118 which is capable of contacting the second edge 110 of the sidewall 106 when the balloon 104 is expanded. The term “end portion” of the balloon 104 refers to that part of the balloon 104 which extends from where the balloon 104 contacts an edge 108, 110 of the stent 102 to the end or terminus of the balloon 104a or 104b. The end portion includes not only the outer surface 112 of the balloon 104 but also what is surrounded by the outer surface 112. Thus, the first end portion 116 of the balloon is that portion which extends from where the first edge 108 of the stent sidewall contacts the balloon 104 to the first end 104a of the balloon, which is axially beyond the first edge 108 of the stent 102. Likewise, the second end portion 118 of the balloon 104 is that portion which extends from where the balloon 104 contacts the second edge 110 of the stent sidewall 106 to the second end 104b of the balloon 104. The term “middle portion” refers to the remainder of the balloon 104 that is between the two end portions of the balloon 104. The “outer surface” of the balloon 104 refers to the surface that is exposed to a body lumen wall. Therefore, the middle portion 114 and two end portions 116, 118 of the balloon 104 can each have an outer surface.

At least the first end portion 116 of the balloon 104 comprises a first biologically active material to be delivered to a portion of the body lumen wall that is adjacent to the first edge 108 of the stent sidewall 106. The balloon 104 is capable of being deployed or expanded in the body lumen so that the first end portion 116 of the balloon 104 extend axially beyond the first edge 108 of the stent sidewall 106 in order to deliver the first biologically active material to a portion of the body lumen wall that is adjacent to the first edge 108 of the stent sidewall 106. For example, when the system is deployed in a body lumen and the balloon is inflated and the stent is in an expanded position, the first end portion of the balloon extends axially beyond the first edge of the stent sidewall and the second end portion of the balloon extends axially beyond the second edge of the stent sidewall. FIGS. 1-7 show the system of the present invention in which the balloon is inflated and the stent is expanded and the end portions of the balloon extend beyond the edges of the stent sidewall. In embodiments, as shown in FIG. 1, where the first end portion and second end portion of the balloon extend axially beyond the edges of the stent sidewall, the balloon can deliver the biologically active material to a larger area than the conventional coated stent and reduce the “edge effects” caused by insufficient biologically active material being released at the edges of the stent.

Preferably, portions of the first end portion and the second end portion of the balloon that comprise the first biologically active material are contiguous with the edges of the stent and in contact with the body lumen wall that is beyond the edges of the stent.

Moreover, the entire end portion of the balloon does not have to comprise the biologically active material. For instance, if the end portion comprises a coating containing the biologically active material, the coating may be disposed only on a part of the end portion.

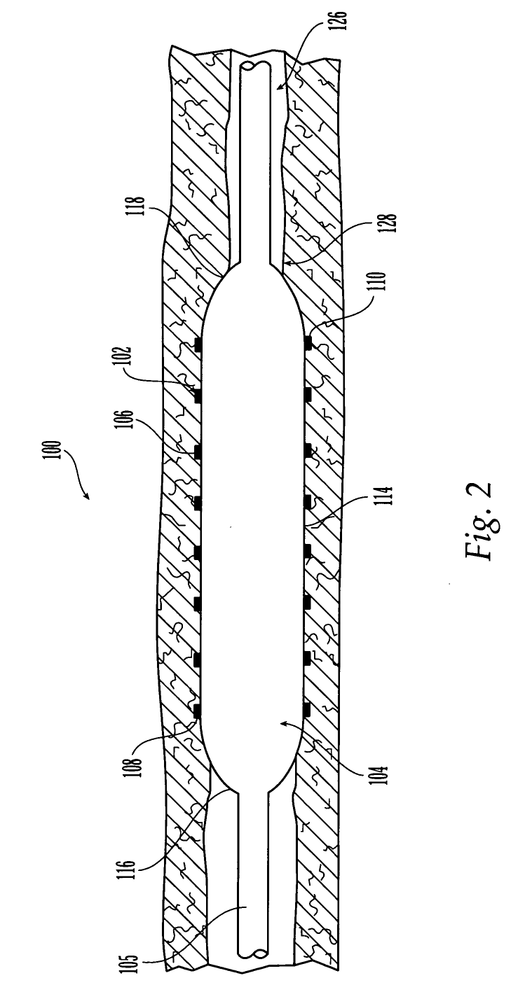

FIG. 2 is a cross-sectional side view of the medical device system 110 of FIG. 1 within a body lumen 126. The first edge 108 of the stent sidewall 106 and the first end portion 116 of the balloon 104 are contiguous and in contact with the body lumen wall 128. Also, the second edge 110 of the stent sidewall 106 and the second end portion 118 of the balloon 104 are contiguous and in contact with the body lumen wall 128 to be treated.

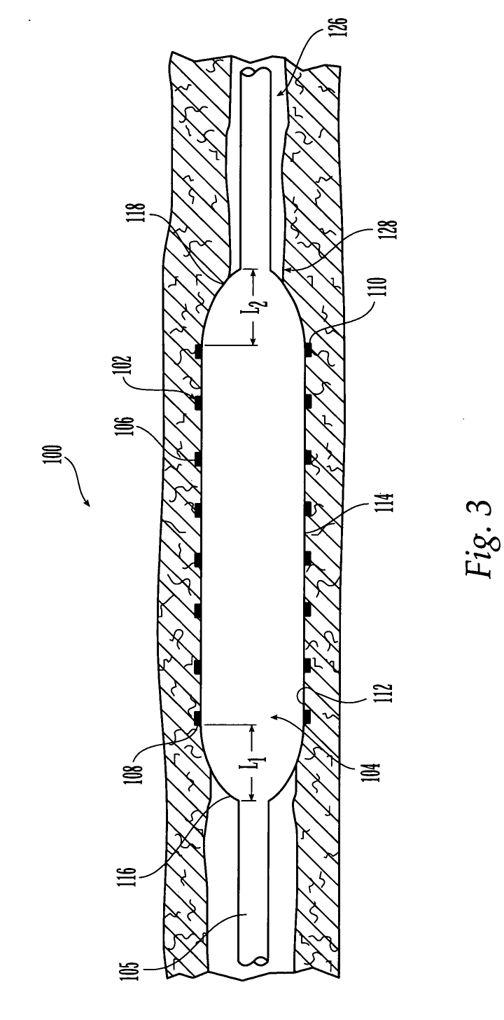

The end portions 116, 118 of the balloon 104 may extend any desired distance from the edges 108, 110 of the stent sidewall 106 as long as the first end portion 116 and/or second end portion 118 are capable of delivering a biologically active material to the body lumen wall that is adjacent to edge of the stent where injury has or may occur. It is not necessary for the first end portion 116 and the second end portion 118 to extend beyond the edges of the stent 102 by the same distance. FIG. 3 is a cross-sectional side view of a medical device system 100 of the present invention deployed within a body lumen in which the first end portion 116 and second end portion 118 of the balloon 104 are different lengths, i.e., L1 is greater than L2.

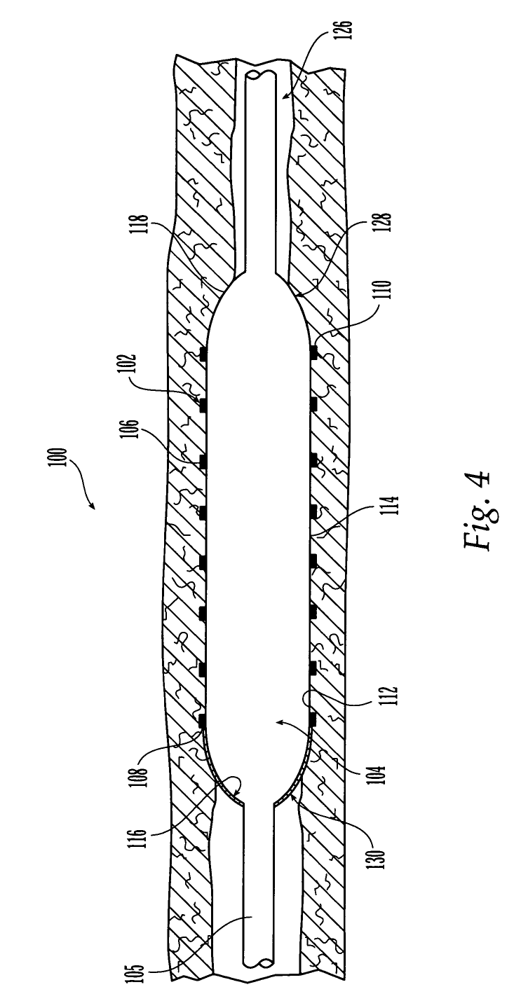

The first biologically active material may be included in a balloon coating 130 on the surface of one or both of the end portions of the balloon 104. FIG. 4 is a cross-sectional side view of a medical device system 100 of the present invention deployed within a body lumen 126 in which the first end portion 116 of the balloon 104 is coated with a balloon coating 130.

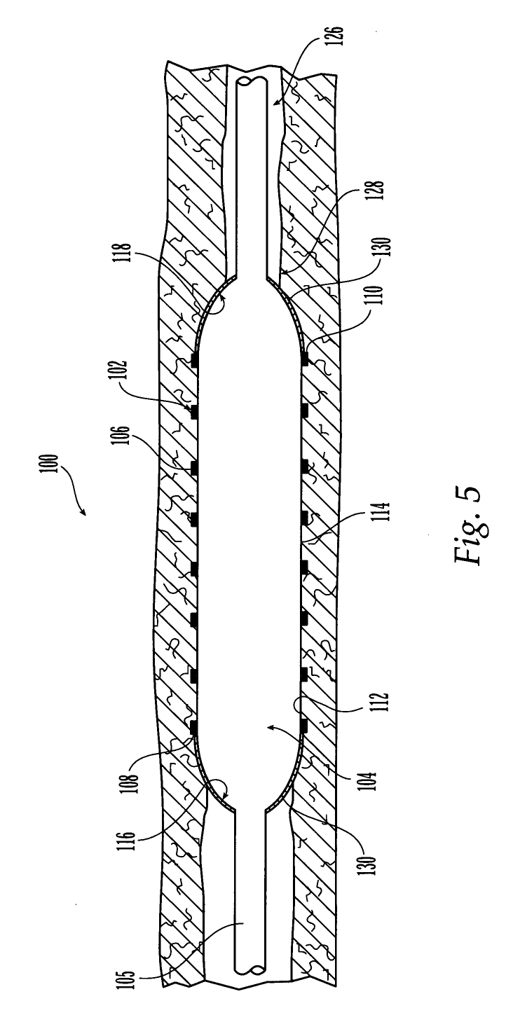

FIG. 5 is a cross-sectional side view of a medical device system 100 of the present invention deployed within a body lumen 126 in which the first end portion 116 and the second end portion 118 are coated with a balloon coating 130. The balloon coating may include a polymer. In addition, a polymeric material may first be applied to the outer surface of the end portions and then a biologically active material may be absorbed or adsorbed into the polymeric material to form a coating. For example, the coating may be formed by dipping a composition comprising the polymer into a composition comprising the biologically active material so that the biologically active material is absorbed or adsorbed into the polymer composition.

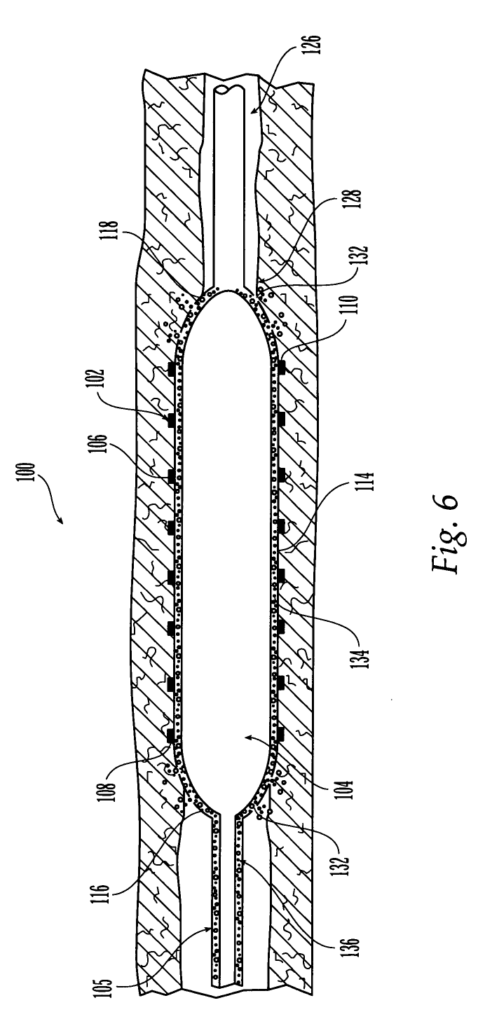

Alternatively, the first biologically active material may be disposed within the balloon and allowed to diffuse out of the balloon through pores created in the balloon. The pores may be holes or channels in the balloon. FIG. 6 is a cross-sectional side view of a medical device system 100 of the present invention deployed within a body lumen 126 in which the first end portion 116 and the second portion 118 of the balloon 104 comprise a plurality of pores 132 through which a biologically active material 134 can be delivered to the body lumen wall 128. The biologically active material 134 can be delivered to the pores 132 of the balloon 104 by using a biologically active material lumen 136 of a balloon catheter 105. As the balloon 104 is expanded, the biologically active material 134 in the catheter lumen 136 can be forced through the pores 132 and delivered to the body lumen wall 128.

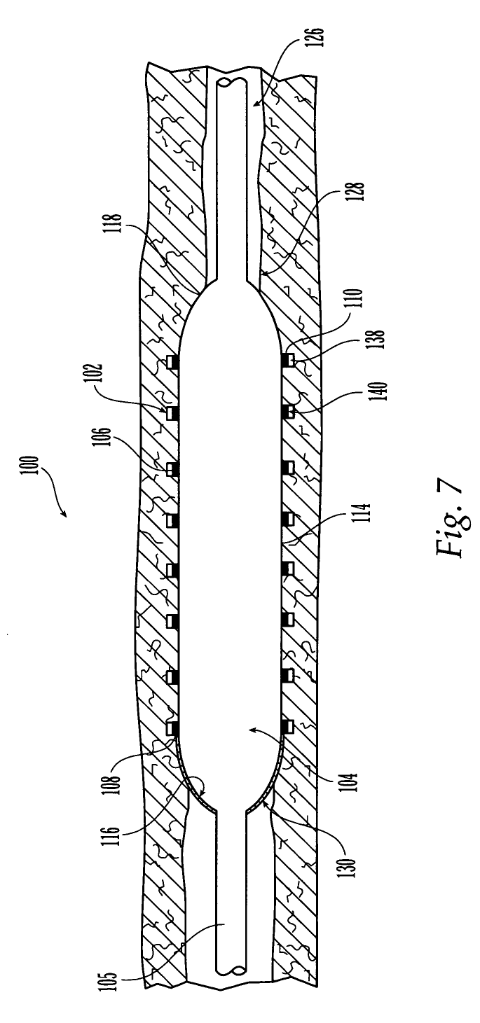

The stent 102 may also comprise a stent coating 138 on the surface 146 of the stent sidewall 106. FIG. 7 is a cross-sectional side view of a medical device system 100 of the present invention deployed within a body lumen 126 in which the first end portion 116 and the second end portion 118 of the balloon 104 are coated with a balloon coating 130 and a surface 138 of the sidewall 106 of the stent 102 is coated with a stent coating 138. The stent coating 138 preferably includes a second biologically active material. The stent coating may also include a polymer. The first biologically active material and the second biologically active material may be the same or different. In addition, the stent coating may contain the same or a different polymer as the balloon coating. Thus, the stent coating and the balloon coating may be the same or different.

In one embodiment, the middle portion of the balloon is substantially free of a biologically active material. In such embodiment, the stent preferably includes a stent coating.

The present invention is also directed to a method of making a medical device system for treating a body lumen wall of a body lumen including providing a stent as described above, and disposing a balloon as described above within the sidewall of the stent. In addition, the balloon is capable of being expanded or deployed so that the first end portion of the balloon extends beyond the first edge of the stent sidewall in order to deliver the first biologically active material to a portion of the body lumen wall that is adjacent to the first edge of the stent sidewall.

Preferably, the stents of the present invention comprise a tubular portion which is insertable into the body of a patient. The tubular portion of the medical device need not be completely cylindrical. For instance, the cross-section of the tubular portion can be any shape, such as rectangle, a triangle, etc., not just a circle. Stents which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. In preferred embodiments, suitable stents include an Express™ stent or an Express2™ stent (Boston Scientific Corp., Natick, Mass.). A bifurcated stent is also included among the medical devices suitable for the present invention. Other medical devices suitable for use with the present invention, as known to one skilled in the art, include, but are not limited to, stent grafts and vascular or other grafts, and vena cava filters. Preferably, a self-expanding stent or balloon expandable stents is used.

The stent may be fabricated from metallic, ceramic, or polymeric materials, or combinations thereof. The material may be porous or nonporous. Porous materials can be microporous, nanoporous or mesoporous. Preferred materials are metallic. Suitable metallic materials include metals and alloys based on titanium (such as nitinol, nickel titanium alloys, thermo memory alloy materials), stainless steel, tantalum, nickel chrome, or certain cobalt alloys including cobalt chromium nickel alloys such as Elgiloy® and Phynox®. Other suitable materials include platinum enhanced radiopaque stainless steel (PERSS®) and thin layer carbon or diamond like coatings. The components may also include parts made from other metals such as, for example, gold, platinum, or tungsten. Metallic materials also include clad composite filaments, such as those disclosed in WO 94/16646.

Suitable ceramic materials include, but are not limited to, oxides of the transition elements such as titanium oxides, titanium nitric oxides, hafnium oxides, iridium oxides, chromium oxides, and aluminum oxides. Silicon based materials may also be used.

The polymer(s) useful for forming the components of the medical devices should be ones that are biocompatible and avoid irritation to body tissue. The polymers can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful include, without limitation, dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

Any suitable balloon intended for use in delivering an expandable, implantable stent or other medical device mounted thereon may be used. As such, the physical characteristics of the balloon may vary. The balloon may be compliant or non-compliant or may be compliant in-part and non-compliant in part. The wall thickness of the balloon may be constant over the entire balloon or may vary in different parts of the balloon. The balloon may be formed of one layer of material or may consist of a plurality of layers. The balloon may be formed of a single piece of balloon material or may be formed of several pieces joined together along the length of the balloon.

The balloon may be made from any balloon material known in the art including polyethylene, polyethylene terephthalate (PET), Arnitel, Hytrel, polyetherether ketone (PEEK), Pebax, Teflon as well as other polyolefins. Other thermoplastic elastomers may be used as well. More generally, any thermoplastic elastomer treatable by a blow molding process may be used.

As discussed above, the balloon comprises a first biologically active material. The balloon may be designed in any suitable configuration to accommodate the biologically active material. For example, the balloon may include channels within the walls of the balloon and outside an inflation balloon for containing the biologically active material. The balloon may further include pores on the outer surface of the first end portion and/or the second end portion through which the biologically active material can pass. The pores may be in the form of holes or channels and may be nano- or micro-sized.

In another embodiment, the balloon includes a balloon coating on the outer surface of at least the first end portion. Preferably, the balloon coating is disposed on the first end portion and the second end portion. The balloon coating is applied before delivery of the medical device system. For example, the balloon coating may be applied to the first and second end portions prior to folding the balloon.

In another embodiment a polymer coating capable of absorbing or adsorbing the first biologically active material is applied to the surface of at least the first end portion of the balloon. Once the stent is mounted on the balloon, the end portions can be exposed such as by dipping into a solution containing the first biologically active material. The polymer and biologically active material should be selected so that sufficient quantities of the biologically active material can be absorbed or adsorbed after a short exposure.

The biologically active material can be applied to the balloon when the balloon is assembled or after balloon has been inserted into the stent or later on by a medical professional shortly before the device is inserted into a patient. The biologically active material may be applied to the outer surface of the balloon, alone or in conjunction with other materials, such as a polymer, to form a coating. For example, the biologically active material can be applied to the outer surface of the first and/or second end portions in a coating composition containing the biologically active material and a polymer, to form a coating. Specifically, a coating composition of biologically active material and a polymer can be prepared and then applied to the outer surface.

The term “therapeutic agent” as used in the present invention encompasses drugs, genetic materials, and biological materials and can be used interchangeably with “biologically active material”. Non-limiting examples of suitable therapeutic agents include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors as well as anti-sense nucleic acid molecules such as DNA, RNA, and RNAi. Viral vectors include adenoviruses, gutted adenoviruses, adeno associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX 015), and hybrid vectors. Non viral vectors include artificial chromosomes and mini chromosomes, plasmid DNA vectors (e.g., pCOR), cationic polymers (e.g., polyethyleneimine, polyethyleneimine (PEI)) graft copolymers (e.g., polyether PEI and polyethylene oxide PEI), neutral polymers PVP, SP1017 (SUPRATEK), lipids or lipoplexes, nanoparticles and microparticles with and without targeting sequences such as the protein transduction domain (PTD).

The term “biological materials” include cells, yeasts, bacteria, proteins, peptides, cytokines and hormones. Examples for peptides and proteins include vascular endothelial growth factor (VEGF), transforming growth factor (TGF), endothelial mitogenic growth factors, transcription factors, proteinkinases, CD inhibitors, fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIP), cytokines, interleukin (e.g., IL-1, IL-2, IL-3, IL-4, IL-S, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mononuclear cells, progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, epothilone D, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, and estrogen derivatives such as estradiol and glycosides.

Also, the biologically active materials of the present invention include nitric oxide adducts, which prevent and/or treat adverse effects associated with use of a medical device in a patient, such as restenosis and damaged blood vessel surface. Typical nitric oxide adducts include nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono- or poly-nitrosylated proteins, particularly polynitrosated albumin or polymers or aggregates thereof. The albumin is preferably human or bovine, including humanized bovine serum albumin. Such nitric oxide adducts are disclosed in U.S. Pat. No. 6,087,479 to Stamler et al. which is incorporated herein by reference.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

A biologically active material may be encapsulated in micro-capsules by the known methods. Preferably, the biologically active material is hydrophobic, e.g., paclitaxel, actinomycin, sirolimus, tacrolimus, everolimus, dexamethasone, and hydrophobic nitric oxide adducts. Preferred biologically active materials also include paclitaxel, a paclitaxel analogue, a paclitaxel derivative, and a combination thereof.

The biologically active material may also be applied with a coating composition. Coating compositions suitable for applying biologically active materials to the devices of the present invention preferably include a polymeric material and a biologically active material dispersed or dissolved in a solvent which does not alter or adversely impact the therapeutic properties of the biologically active material employed. Coating compositions may be applied by any method to the surface of the balloon to form a coating. Examples of suitable methods include, but are not limited to, spraying such as by conventional nozzle or ultrasonic nozzle, dipping, rolling, electrostatic deposition, and a batch process such as air suspension, pancoating or ultrasonic mist spraying. Also, more than one coating method may be used. Coating compositions suitable for applying a coating to the balloons of the present invention may include a polymeric material dispersed or dissolved in a solvent suitable for the balloon wherein upon applying the coating composition to the balloon, the solvent is removed. Such methods are commonly known to the skilled artisan.

The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material are styrene isobutylene styrene copolymers. Other polymers that may be used include ones that may be dissolved and cured or polymerized or polymers having relatively low melting points that can be blended with biologically active materials. Additional suitable polymers include thermoplastic elastomers in general, polyolefins, polyisobutylene, ethylene alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene methyl methacrylate copolymers, acrylonitrile styrene copolymers, ABS (acrylonitrile butadiene styrene) resins, ethylene vinyl acetate copolymers, polyamides such as Nylon 66 and polycaprolactone, alkyd resins, polycarbonates, polyoxymethylenes, polyimides, polyethers, epoxy resins, rayon triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid polyethylene oxide copolymers, EPDM (ethylene propylene diene) rubbers, fluorosilicones, polyethylene glycol, polysaccharides, phospholipids, and combinations of the foregoing.

Preferably, polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the stent is subjected to forces, stress or mechanical challenge, such as by expansion and contraction.

Preferred polymers of the balloon coating are biostable polymers capable of releasing the biologically active material. Such polymers include, but are not limited to, hydrogel or phosphoryl choline.

Solvents used to prepare coating compositions include ones which can dissolve or suspend the polymeric material in solution. Examples of suitable solvents include, but are not limited to, tetrahydrofuran, methylethylketone, chloroform, toluene, acetone, isooctane, 1,1,1, trichloroethane, dichloromethane, isopropanol, IPA, and mixtures thereof.

Coating compositions may be used to apply one type of biologically active material or a combination of biologically active materials. In general, the coating layer may be applied as one homogeneous layer, however, the coating layer may be composed of a plurality of layers comprised of different materials. If the coating layer is composed of a plurality of layers, each layer may contain a single biologically active material or a combination of biologically active materials. In addition, one or more layers may be free of a biologically active material.

A controlled-release coating of a biologically active material may be prepared by a coating composition comprising an appropriate hydrophobic polymer. For example, a controlled-release coating may comprise a coating layer containing a biologically active material and a top coating layer comprising a hydrophobic polymer. Also, a controlled-release coating may be prepared from a coating composition containing a mixture of a hydrophobic polymer and a biologically active material.

The amount of the polymeric material present in the coatings can vary based on the application for the medical device system. One skilled in the art is aware of how to determine the desired amount and type of polymeric material used in the coating, and the desired thickness of the coating.

As described above, a stent coating may be disposed on a surface of the stent. The stent coating on the stent may contain a second biologically active material such as those described above. The biologically active material may also be applied with a coating composition. Coating compositions suitable for applying biologically active materials to the devices of the present invention preferably include a polymeric material and a biologically active material dispersed or dissolved in a solvent which does not alter or adversely impact the therapeutic properties of the biologically active material employed. Suitable polymers and solvents include, but are not limited to, those listed above. Coating compositions may be applied by any method to a surface of a stent to form a coating including, but not limited to, those described above with respect to the balloon coating. The first biologically active material of the balloon may be the same as or different than the second biologically active material in the stent coating.

The system of the present invention may be deployed within a body lumen by any suitable method as known to one skilled in the art. The system may be delivered with a catheter. For example, a catheter having a balloon mounted thereon may also be used with the system of the present invention. Any suitable balloon-based stent delivery catheter may be used, such as rapid-exchange catheters and fixed wire catheters. A balloon catheter known to artisans can be used for the invention, e.g., the balloon catheters disclosed in U.S. Pat. Nos. 5,746,745, 5,693,014, 6,010,480 and 6,033,381. Other suitable catheters for use in the present invention include those disclosed in WO 98/07390. In addition, a self-expanding stent disposed about a balloon which is retained in its place by a retractable sheath may be used.

The exact configuration of the delivery apparatus will depend on what other functions are desired. For example, those of ordinary skill in the art will recognize how to accommodate balloon expandable stents, stent-grafts, grafts and vena cava filters. In addition, any other suitable device having a balloon thereon for delivery of any of the above expandable, implantable stents may also be used.

A balloon catheter assembly and a guidewire may be used with the system of the present invention. The balloon and stent may be deployed simultaneously. For example, the stent may be collapsed to a small diameter, placed over an angioplasty balloon catheter and moved into the area to be treated. The balloon catheter will be inflated to expand the stent. Thus, the balloon and stent are delivered in a compressed state to a body lumen to be treated, and then expanded against the body lumen wall.

In addition to preventing the onset of restenosis, the system of the present invention may be used to treat restenosis that has already been diagnosed in the areas adjacent to the ends of previously deployed stents. Thus, for example, a balloon may be deployed within the stent that was deployed in a previous, separate procedure to form the medical device system of the present invention. Such medical device systems may be temporarily inserted into or semi-permanently or permanently implanted in the body of a patient. The disclosed system may be used to deliver a therapeutic agent to various types of body lumina.

It should be appreciated that the features and components described herein may be used singly or in any combination thereof. Moreover, the present invention is not limited to only the embodiments specifically described herein, and may be used with medical devices other than stents. The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

While the foregoing description and drawings may represent preferred embodiments of the present invention, it should be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, and proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and not limited to the foregoing description.

We claim:

1. A medical device system for treating a body lumen wall of a body lumen comprising:
a stent that is insertable into a body lumen having a body lumen wall, wherein the stent comprises a sidewall having a first edge and a second edge; and
a balloon capable of being disposed within the stent sidewall, wherein the balloon has an outer surface, a middle portion, a first end portion that is capable of contacting the first edge of the stent sidewall when the balloon is expanded, and a second end portion that is capable of contacting the second edge of the stent sidewall when the balloon is expanded,
wherein at least the first end portion of the balloon comprises a first biologically active material, and the balloon is capable of being expanded in the body lumen so that the first end portion of the balloon extends axially beyond the first edge of the stent sidewall in order to deliver the first biologically active material to a portion of the body lumen wall that is adjacent to the first edge of the stent sidewall.

2. The system of claim 1, wherein the first end portion of the balloon comprises pores through which the biologically active material can be delivered to the body lumen wall.

3. The system of claim 1, wherein the balloon further comprises a balloon coating disposed on the outer surface of at least the first end portion of the balloon, and the balloon coating comprises the first biologically active material.

4. The system of claim 3, wherein the balloon coating further comprises a polymer.

5. The system of claim 4, wherein the polymer is a biostable polymer capable of releasing the biologically active material.

6. The system of claim 5, wherein the polymer comprises a hydrogel or phosphoryl choline.

7. The system of claim 1, wherein the first biologically active material comprises paclitaxel, a paclitaxel analogue, a paclitaxel derivative, or a combination thereof.

8. The system of claim 1, wherein the second end portion of the balloon comprises the first biologically active material, and the balloon is capable of being expanded so that the second end portion of the balloon extends axially beyond the second edge of the stent sidewall in order to deliver the first biologically active material to a portion of the body lumen wall that is adjacent to the second edge of the stent sidewall.

9. The system of claim 1, wherein the stent sidewall further comprises a surface and a stent coating disposed on at least a portion of the surface of the stent sidewall.

10. The system of claim 9, wherein the stent coating comprises a second biologically active material.

11. The system of claim 10, wherein the second biologically active material comprises an antiproliferative agent.

12. The system of claim 10, wherein the stent coating further comprises a polymer.

13. A medical device system for treating a body lumen wall of a body lumen comprising:
a stent that is insertable into a body lumen having a body lumen wall, wherein the stent comprises a sidewall having a surface, a first edge and a second edge, and a stent coating disposed on at least a portion of the surface of the stent sidewall; and
a balloon capable of being disposed within the stent sidewall, wherein the balloon has an outer surface, a middle portion, a first end portion that is capable of contacting the first edge of the stent sidewall when the balloon is expanded, and a second end portion that is capable of contacting the second edge of the stent sidewall when the balloon is expanded,
wherein at least the first end portion of the balloon and the second end portion of the balloon both comprise a first biologically active material, and the balloon is capable of being expanded so that the first end portion of the balloon extends axially beyond the first edge of the stent sidewall and the second end portion of the balloon extends axially beyond the second edge of the stent sidewall in order to deliver the first biologically active material to portions of the body lumen wall that are adjacent to the first edge and the second edge of the stent sidewall, respectively.

14. The system of claim 13, wherein the first end portion of the balloon and the second end portion of the balloon both comprise pores through which the biologically active material can be delivered to the body lumen wall.

15. The system of claim 13, wherein the balloon further comprises a balloon coating disposed on both the outer surface of the first end portion of the balloon and the outer surface of the second end portion of the balloon, and the balloon coating comprises the first biologically active material.

16. The system of claim 15, wherein the balloon coating further comprises a polymer.

17. The system of claim 16, wherein the polymer comprises a biostable polymer capable of releasing the biologically active material.

18. The system of claim 17, wherein the polymer comprises a hydrogel or phosphoryl choline.

19. The system of claim 15, wherein the first biologically active material comprises paclitaxel, a paclitaxel analogue, a paclitaxel derivative, or a combination thereof.

20. The system of claim 13, wherein the stent coating comprises a second biologically active material.

21. The system of claim 20, wherein the second biologically active material comprises an antiproliferative agent.

22. The system of claim 21, wherein the stent coating further comprises a polymer.

23. A medical device system for treating a body lumen wall of a body lumen comprising:
a stent that is insertable into a body lumen having a body lumen wall, wherein the stent comprises a sidewall having a first edge and a second edge; and
a balloon capable of being disposed within the stent sidewall, wherein the balloon has an outer surface, a middle portion, a first end portion that is capable of contacting the first edge of the stent sidewall when the balloon is expanded, and a second end portion that is capable of contacting the second edge of the stent sidewall when the balloon is expanded,
wherein at least the first end portion of the balloon comprises a first biologically active material, and the balloon is capable of being expanded so that the first end portion of the balloon extends axially beyond the first edge of the stent sidewall in order to deliver the first biologically active material to a portion of the body lumen wall that is adjacent to the first edge of the stent sidewall, and the middle portion of the balloon is substantially free of a biologically active material.

24. The system of claim 23, wherein the first end portion of the balloon comprises pores through which the biologically active material can be delivered to the body lumen wall.

25. The system of claim 23, wherein the balloon further comprises a balloon coating disposed on the outer surface of at least the first end portion of the balloon, and the balloon coating comprises the first biologically active material.

26. The system of claim 25, wherein the balloon coating further comprises a polymer.

27. The system of claim 26, wherein the polymer comprises a biostable polymer capable of releasing the biologically active material.

28. The system of claim 27, wherein the polymer comprises a hydrogel or phosphoryl choline.

29. The system of claim 23, wherein the first biologically active material comprises paclitaxel, a paclitaxel analogue, a paclitaxel derivative, or a combination thereof.

30. The system of claim 23, wherein the second end portion of the balloon comprises the first biologically active material, and the balloon is capable of being expanded so that the second end portion of the balloon extends axially beyond the second edge of the stent sidewall in order to deliver the first biologically active material to a portion of the body lumen wall that is adjacent to the second edge of the stent sidewall.

31. The system of claim 23, wherein the stent sidewall further comprises a surface and a stent coating is disposed on at least a portion of the surface of the stent sidewall.

32. The system of claim 31, wherein the stent coating comprises a second biologically active material.

33. The system of claim 32, wherein the second biologically active material comprises an antiproliferative agent.

34. The system of claim 32, wherein the stent coating further comprises a polymer.

35. A method of making a medical device system for treating a body lumen wall of a body lumen comprising:
providing a stent that is insertable into a body lumen having a body lumen wall, wherein the stent comprises a sidewall having a first edge and a second edge;
disposing a balloon within the sidewall, wherein the balloon has an outer surface, a middle portion, a first end portion that is capable of contacting the first edge of the stent sidewall when the balloon is expanded and a second end portion that is capable of contacting the second edge of the stent sidewall when the balloon is expanded,
wherein the first end portion of the balloon comprises a first biologically active material, and the balloon is capable of being expanded so that the first end portion of the balloon extends axially beyond the first edge of the stent sidewall in order to deliver the first biologically active material to a portion of the body lumen wall that is adjacent to the first edge of the stent sidewall.

36. The method of claim 35, wherein the second end portion of the balloon comprises the first biologically active material, and the balloon is capable of being expanded so that the second end portion of the balloon extends axially beyond the second edge of the stent sidewall in order to deliver the first biologically active material to a portion of the body lumen wall that is adjacent to the second edge of the stent sidewall.
